(19) United States
(12) Patent Application Publication
(10) Pub. No.: US 2014/0024582 A1
(43) Pub. Date: Jan. 23, 2014

(54) STABLE INSULIN FORMULATIONS AND METHODS OF MAKING AND USING THEREOF

(71) Applicant: Genecopoeia, Inc., Rockville, MD (US)

(72) Inventor: Guohan YANG, Mansfield, MA (US)

(73) Assignee: GENECOPOEIA, INC., Rockville, MD (US)

(21) Appl. No.: 13/848,617

(22) Filed: Mar. 21, 2013

Related U.S. Application Data
(63) Continuation of application No. 13/519,552, now abandoned.
(60) Provisional application No. 61/293,738, filed on Jan. 11, 2010.

(30) Foreign Application Priority Data
Jul. 22, 2010 (CN) 201010244678.6

(51) Int. Cl. A61K 47/18; A61K 38/28

(52) U.S. Cl. CPC A61K 47/183; A61K 38/28
USPC 514/6.4; 514/5.9

(57) ABSTRACT

The invention provides methods for improving the physical and chemical stabilities of therapeutic proteins in solutions by reducing or eliminating protein degradation, aggregation and precipitation using small molecule stabilizing agents such as proline, arginine and/or compounds capable of forming a Schiff bond with amino groups of the protein. The invention further provides liquid formulations of a monomeric insulin stabilized by one of more stabilizing agents such as proline, arginine and/or acetone. Also provided are methods of making and using the stabilized monomeric insulin formulations.

### CROSS-REFERENCE TO RELATED APPLICATIONS

This application claims the benefits of U.S. Provisional Application No. 61/293,738, filed on Jan. 11, 2010, and Chinese Patent Application No. 201010244678.6, filed on Jul. 22, 2010, which are incorporated herein by reference in their entireties.

### FIELD OF THE INVENTION

This invention relates to use of small molecule chemical reagents in aqueous formulation of proteins, including therapeutic proteins, to minimize and/or eliminate protein precipitation caused by protein denaturing (e.g. fibrillation), and to minimize covalent protein polymers formed through transamidation or disulfide scrambling reactions. This invention also relates to therapeutic protein solutions containing small molecule stabilizing agents, thus having improved on-shelf storage stability, and methods for manufacturing of such stable protein solutions and their therapeutic uses.

### BACKGROUND OF THE INVENTION

With the well-developed DNA recombinant technology, more and more proteins are being developed as therapeutic pharmaceuticals. Development of stable protein formulations is one of the critical steps in developing protein as a therapeutic product. Extensive research has been conducted regarding protein stability. See, e.g., Pearlman, R. and Wang, Y. J., “Formulation, characterization, and stability of protein drugs” Plenum Press, New York (1996); and Chang, B. S. and Hershenson, S., Chapter 1, “Practical Approaches to Protein Formulation Development, Rational Design of Stable Protein Formulations” edited Carpenter and Manning Eds., Kluwer Academic & Plenum Publishers, New York (2002). Besides various chemical reactions, such as disulfide scrambling, deamidation, peptide cleavage, oxidation, a major instability problem relates to non-covalent aggregation that is often immunogenic and sometimes produces precipitates.

Proteins are often formulated into solutions or solids (lyophilized, spray-dried, or spray-freeze-dried) together with excipients to maximize stability during manufacturing and storage. In liquid formulations, optimization of pH, ionic additives, amino acids, saccharides, surfactants, protein concentrations and raw material purity may provide solutions to the aggregation problem.

A number of strategies have been employed to improve the property and stability of therapeutic proteins. One examples is protein modification with hydrophilic polymers, e.g., polyalkylene oxides (Roberts et al., Adv. Drug Rev. 54:459-476 (2002)), polysaccharides like polysialic acid (Fernandes et al., Biochim. Biophys. Acta 1341:26-34 (1997)), dextrans, or hydroxyl alkyl starch. Poly(ethylene glycol) (PEG) derivatized therapeutic proteins are known, e.g., interferon a-2 (Peleg-Shulman, et al., J. Med. Chem. 47:4897-4904 (2004)), exendin-4 (Tsubery, et al., J. Biol. Chem. 37:38118-38124 (2004)), and interferon-β-1b (Zhao, et al., Bio-conjugate Chem. 17:341-351 (2006)). Chemical modification using polyethylene glycol has been used to improve protein stability (US Patent No. 6,890,518). However, chemical modification of protein and peptide drugs often leads to reduced activity of the protein or peptide.

Chaperones have been reported to reduce or prevent protein aggregation that can often lead to precipitation. See Ben-Zvi, A. et al., J. Biol. Chem. 279(36):37298-37303 (2004). Congo Red conjugate which binds to both FK506 binding protein and beta-amyloid peptide has been reported to hinder amyloid fibrillation. See Gestwicki, J. E. et al., Science, 306:865-869 (2004).

Another strategy has been to use amino acids as excipients in protein formulations for various benefits, mostly for ionic strength and buffering reagents. Arginine was used in the process of protein refolding to enhance recovery as a guanidine substitute. See Shiraki, K. et al., J. Biochem. 132:591-595 (2002). Proline has been found to increases solubility of urea-denatured protein inclusion body, but it hinders refolding efficiency. See Samuel, D. et al., Biochem. Mol. Biol. Int. 41(2):235-42 (1997); Protein Science, 9(2):344-352 (2000).

Insulin is a small protein used medically to treat some forms of diabetes mellitus. Although insulin is produced and stored in the body as a hexamer, the active form is the monomer. While the hexamer is more stable and suitable for storage, the monomer is a much faster reacting drug, which gives diabetics more flexibility in their daily schedule. One major problem for insulin formulation is fibrillation, which leads to aggregation and precipitation. Several insulin formulations (See, e.g., U.S. Pat. Nos. 5,070,186, 6,211,144, 6,489,292, 6,652,885, 6,734,162, 6,852,694, 6,960,561, 7,021,309, 7,279,457, 7,490,603, and 7,648,960) aimed to provide fast acting insulin therapies showed marginal improvement over hexameric zinc insulin. There remains a need for a fast acting insulin therapy with improved stability.

### BRIEF SUMMARY OF THE INVENTION

This invention provides a monomeric insulin formulation and methods for making and using the formulation. The method used for stabilizing the insulin monomers are applicable in stabilizing other small therapeutic proteins that fibrillate and precipitate like monomeric insulin, such as calcitonin, parathyroid hormone (PTH), human growth hormone, and glucagon.

In one aspect, the invention provides a stable liquid insulin composition comprising a monomeric insulin and proline, a proline derivative, and/or arginine. In some embodiments, the concentration of proline in the composition is about 0.9 to about 7 mol/L. In some embodiments, the concentration of proline in the composition is about 1 to about 7 mol/L. In some embodiments, the concentration of arginine in the composition is about 0.4 mol/L or higher. In some embodiments, the concentration of arginine in the composition is about 0.4 mol/L to about 2 mol/L. In some embodiments, the monomeric insulin is a human insulin, a porcine insulin, a bovine insulin, an insulin analog (e.g. insulin lispro, insulin aspart, insulin glulisine, other insulin variants), an insulin derivative (e.g. an insulin chemically derivatized with a poly(ethylene glycol) and/or a fatty acid), or a combination thereof in any ratio. In some embodiments, the insulin is a zinc-free human insulin, a zinc-free porcine insulin, a zinc-free bovine insulin, a zinc-free insulin analog, a zinc-free insulin derivative, or a combination thereof in any ratio. In some embodiments, the insulin may be extracted from a living tissue, chemically synthesized, or prepared using a recombinant technology. In some embodiments, the concentration of monomeric insulin in the composition is about 0.01 mg/mL to about 10 mg/mL.

In some embodiments, the stable liquid insulin composition further comprises a stabilizing agent which is a compound capable of forming a Schiff bond with an amino group. The stabilizing agent is selected from the group consisting of acetone, pyruvic acid, glyoxalic acid, alpha-ketobutyric acid, alpha-ketoglutaric acid, acetoacetic acid, pyridoxal, and pyridoxal pyrophosphate. In some embodiments, the stable liquid insulin composition further comprises acetone. In some embodiments, the stable liquid insulin composition contains about 0 to 50 mg/ml of the stabilizing agent. In some embodiments, the stable liquid insulin composition comprises more than one stabilizing agent.

In some embodiments, the stable liquid insulin composition further comprises a pharmaceutically acceptable excipient, such as glycerol (e.g., 0-50 mg/mL) and/or a phenolic compound (e.g., phenol and/or m-cresol). The composition may contain at least 3 moles of the phenolic compound for every 6 moles of the insulin monomer, up to a sufficient level for hygienic purposes (e.g., 5 mg/mL). In some embodiments, the molar concentration of the phenolic compound is at least 50% of the molar concentration of the insulin monomer in the composition.

In some embodiments, the pH range of the stable liquid insulin composition is about 6.0 to about 8.0, preferably about 6.8 to about 7.8.

In some embodiments, the invention provides a therapeutic formulation of monomeric insulin solution, comprising: an insulin, a stabilizing reagent such as proline or a proline derivative (1-7 mol/L), and a pharmaceutically acceptable excipient; wherein the pH range is between 6.0 and 8.0, preferably between 6.8 and 7.8.

In some embodiments, the invention provides a stabilized therapeutic solution of monomeric insulin that may be used as a therapeutic reagent by means of nasal and/or pulmonary inhalation, syringe injection, insulin pen and/or cartridge, insulin pump. This solution comprises an insulin, a stabilizing reagent such as proline or a proline derivative (1-7 mol/L), and a pharmaceutically acceptable excipient. The pH range of the solution is between 6.0 and 8.0, preferably between 6.8 and 7.8.

In some embodiments, the invention provides a therapeutic formulation of monomeric insulin solutions that lowers the blood glucose level faster than a therapeutic formulation of hexameric zinc-insulin in solution. This formulation comprises an insulin, a stabilizing reagent such as proline or a proline derivative (1-7 mol/L), and a pharmaceutically acceptable excipient. The pH range of the solution is between 6.0 and 8.0, preferably between 6.8 and 7.8.

In another aspect, the invention provides a method of making a stable monomeric insulin solution comprising: (a) mixing a zinc insulin in a solution with a zinc chelating reagent, wherein the molar ratio of the zinc chelating reagent to zinc is from about 1:1 to about 100:1; and (b) adding proline, a proline derivative, and/or arginine to the solution formed in step (a) to form a stabilized insulin solution. In some embodiments, the pH range of the stabilized insulin solution is about 6.0 to about 8.0, preferably about 6.8 to about 7.8. In some embodiments, the zinc insulin is a zinc human insulin, a zinc porcine or bovine insulin, a zinc insulin analog, a zinc insulin derivative, or a combination thereof in any ratio. In some embodiments, the zinc chelating reagent is EDTA or ETPA. In some embodiments, the zinc insulin is extracted from a living tissue, chemically synthesized, or prepared using a recombinant technology. In some embodiments, the stabilized insulin solution contains about 0.9 to about 7 mol/L of proline or proline derivative and/or about 0.4 to about 2.0 mol/L of arginine. In some embodiments, the stabilized insulin solution contains about 1 to about 7 mol/L of proline or a proline derivative. In some embodiments, the stabilized insulin solution contains about 0.01 mg/mL to about 10 mg/mL of monomeric insulin. In some embodiments, the method further comprises adding acetone to the solution formed in step (a) or the stabilized insulin solution formed in step (b).

In another aspect, the invention provides a medicament comprising a monomeric insulin, proline, a proline derivative, and/or arginine, and a pharmaceutically acceptable excipient, wherein the medicament is a liquid suitable for administration in a subject in need thereof by means of nasal and/or pulmonary inhalation, syringe injection, insulin pen and/or cartridge, or insulin pump.

Also provided is a kit, comprising a medicament described herein and a container (e.g. a nasal and/or pulmonary inhaler, an injection syringe, an insulin pen and/or cartridge, or an insulin pump) for holding the medicament. The kit may further comprise instructions for administrating the medicament in a subject in need thereof by nasal and/or pulmonary inhalation, syringe injection, insulin pen and/or cartridge, or insulin pump.

In another aspect, the invention provides a method for lowering blood glucose level or preventing the onset of a hyperglycemic effect in a subject in need thereof comprising administering to the subject an effective amount of a stable liquid insulin composition described herein or a liquid insulin medicament described herein.

In yet another aspect, the invention provides a method for stabilizing a small protein (molecular weight under 20,000 Daltons, e.g., an insulin monomer, calcitonin, PTH, a human growth hormone, or glucagon) in solution comprising mixing the protein in solution with one or more small molecule excipients, such as proline or a proline derivative (e.g., about 1 to about 7 mol/L or about 0.9 to about 7 mol/L), arginine (e.g., about 0.4 to about 2 mol/L), and/or acetone. The small molecule excipients stabilize the protein by minimizing protein precipitation caused by denaturing and/or fibrillation. The small molecule excipients also minimize chemical degradation of the protein.

In some embodiments, the invention provides a method for improving the physical and chemical stability of monomeric insulin in solution, in which proline or a proline derivative is added as the stabilizer at concentrations ranging from 1 to 7 mol/L.

It is to be understood that one, some, or all of the properties of the various embodiments and variations described herein may be combined to form other embodiments and variations of the present invention. These and other aspects of the invention will become apparent to one of skill in the art.

### BRIEF DESCRIPTION OF THE DRAWINGS

FIG. 1 shows a scheme illustrating formation of a stabilized solution of an insulin monomer.

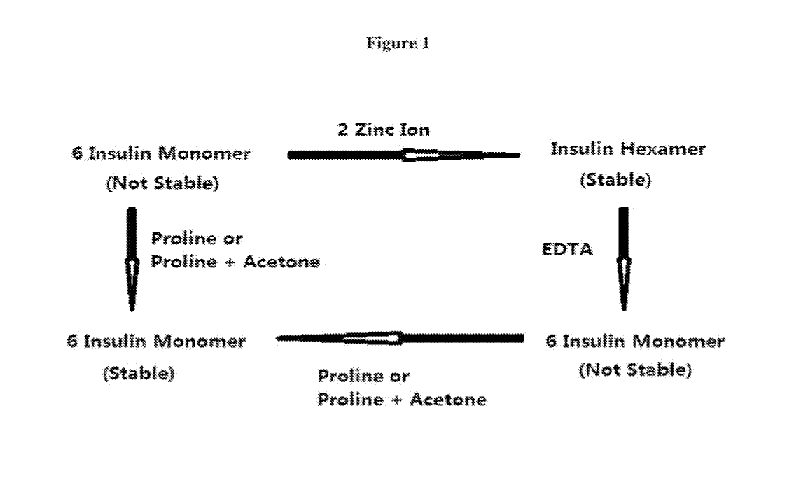

### DETAILED DESCRIPTION OF THE INVENTION

The invention provides a stable liquid protein composition (such as insulin composition) comprising a protein (such as a monomeric insulin) and a stabilizing agent such as proline, a proline derivative, arginine and/or acetone. Methods for making and using the compositions are also provided.

### Definitions

As used herein, “insulin” refers to human insulin, porcine insulin, bovine insulin, insulin derivatives such us insulin chemical modification by fatty acid or poly(ethylene glycol), and insulin analogs of sequence alteration such as insulin lispro, insulin aspart, insulin glulisine, and other variants described herein. As used herein, the term “monomeric insulin” refers to fully dissociated single-molecule insulin. In some embodiments, the term “monomeric insulin” also refers to non-covalently associated insulin dimers or higher oligomers that readily dissociate back to single molecules. In some embodiments, “monomeric insulin” includes single molecular insulin or non-covalent insulin dimers and non-covalent insulin oligomers that are not in the form of chelated complex with metal ions such as zinc.

As used herein, the term “zinc free insulin” refers to insulin bulk material which does not contain zinc ions that form insulin-zinc complex.

A “subject” or an “individual” is a mammal, more preferably a human. Mammals include, but are not limited to, farm animals, sport animals, pets (such as cats, dogs, and horses), primates, mice and rats.

It should be noted that, as used herein, the singular form “a”, “an”, and “the” includes plural references unless indicated otherwise.

When “about” is used to describe a range, the term applies to both lower and upper value of a range. For example, “about X to Y” means “about X to about Y”. Reference to “about” a value or parameter herein includes (and describes) embodiments that are directed to that value or parameter per se. For example, description referring to “about X” includes description of “X.”

It is understood that aspect and embodiments of the invention described herein include “consisting” and/or “consisting essentially of aspects and embodiments.

### Insulin Monomers

Insulin described herein includes natural insulin and insulin analogs and derivatives. Natural insulin is synthesized from insulin preproprotein. After removal of the precursor signal peptide, proinsulin is post-translationally cleaved into three peptides: the B chain and A chain peptides, which are covalently linked via two disulfide bonds to form insulin, and C-peptide. Binding of insulin to the insulin receptor (INSR) stimulates glucose uptake.

The human insulin is composed of two peptide chains, chain A (21 mer) and chain B (30 mer).

A Chain (SEQ ID NO: 1):
GIVEQ CCTSI CSLYQ LENYC N
B Chain (SEQ ID NO: 2):
FVNQH LCGSH LVEAL YLVCG ERGFF YTKPT

Cys(A6) and Cys(A11) form a disulfide bond, Cys(A7) and Cys(B7) form a second disulfide bond, Cys(A20) and Cys(B19) form the third disulfide bond.

The bovine insulin is also a small protein of 51 amino acid and has a structure similar to the human insulin with only a few mutations of amino acids in the sequence:

A Chain (SEQ ID NO: 3):
GIVEQ CCASV CSLYQ LENYC N
B Chain (SEQ ID NO: 4):
FVNQH LCGSH LVEAL YLVCG ERGFF YTPKA

Cys(A6) and Cys(A11) form a disulfide bond, Cys(A7) and Cys(B7) form a second disulfide bond, Cys(A20) and Cys(B19) form the third disulfide bond.

The porcine insulin is also a small protein of 51 amino acid and has a structure similar to human insulin with only one mutation of amino acid at the C-terminus of the B Chain.

A Chain (SEQ ID NO: 1):
GIVEQ CCTSI CSLYQ LENYC N
B Chain (SEQ ID NO: 5):
FVNQH LCGSH LVEAL YLVCG ERGFF YTPKA

Cys(A6) and Cys(A11) form a disulfide bond, Cys(A7) and Cys(B7) form a second disulfide bond, Cys(A20) and Cys(B19) form the third disulfide bond.

Humalog® (insulin lispro, rDNA origin) is a human insulin analog that is a rapid-acting, blood glucose-lowering agent. Chemically, it is Lys(B28), Pro(B29) human insulin analog, created when the two amino acids at positions 28 and 29 on the human insulin B-chain are reversed. (www.rxlist.com/humalog-drug.htm)

NovoLog (insulin aspart [rDNA origin] injection) is a rapid-acting human insulin analog. NovoLog is homologous with regular human insulin with the exception of a single substitution of the amino acid proline by aspartic acid in position B28. (www.rxlist.com/novolog-drug.htm)

APIDRA® (insulin glulisine [rDNA origin] injection) is a rapid-acting human insulin analog used to lower blood glucose. Insulin glulisine differs from human insulin in that the amino acid asparagine at position B3 is replaced by lysine and the lysine in position B29 is replaced by glutamic acid. (www.rxlist.com/apidra-drug.htm)

Insulin degludec is a new generation ultra-long acting basal insulin analogue in clinical development. Degludec retains the human insulin amino acid sequence except for the deletion of Thr(B30) and the addition of a 16-carbon fatty diacid attached to Lys(B29) via a glutamic acid spacer. Formation of insulin multihexamers at the site of subcutaneous injection is the major contributing mechanism to the ultra long duration of action of this new class of insulin analogue (Jonassen I. et al. “Insulin degludec: multi-hexamer formation is the underlying basis for this new generation ultra-long acting basal insulin” Presentation No. 972, 46th EASD Meeting, Stockholm, Sweden, Sep. 20-24, 2010.).

Insulin described herein also includes insulin analogs and/or derivatives, which are variants of any of insulin monomers described herein. Variants may include one, two, three, or more amino acid substitutions, deletions or additions in the A chain and/or the B chain. Variants may be from natural mutations or human manipulation. Changes can be of a minor nature, such as conservative amino acid substitutions that do not significantly affect the activity of the protein. Recombinant DNA technology known to those skilled in the art can be used to create novel mutant proteins or mutants including single or multiple amino acid substitutions, deletions, or additions. Such modified polypeptides can show, e.g., enhanced activity, increased stability, or decreased activity/stability. In addition, they may be purified in higher yields and show better solubility than the corresponding natural polypeptide, at least under certain purification and storage conditions. Variants also include those having one or more amino acid residues deleted, added, or substituted to generate polypeptides that are better suited for expression, scale up, etc., in the host cells chosen. In some embodiments, amino acid sequences of the variants are at least about any of 70%, 75%, 80%, 85%, 90%, 95%, 96%, 97%, 98%, or 99% identical to a insulin protein described herein. In some embodiments, the A chain amino acid sequence of the variants is at least about any of 70%, 75%, 80%, 85%, 90%, 95%, 96%, 97%, 98%, or 99% identical to the amino acid sequence shown in SEQ ID NO:1. In some embodiments, the B chain amino acid sequence of the variants is at least about any of 70%, 75%, 80%, 85%, 90%, 95%, 96%, 97%, 98%, or 99% identical to the amino acid sequence shown in SEQ ID NO:2.

Two polypeptide sequences are said to be “identical” if the sequence of amino acids in the two sequences is the same when aligned for maximum correspondence as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, WI), using default parameters. This program embodies several alignment schemes described in the following references: Dayhoff, M.O. (1978) A model of evolutionary change in proteins—Matrices for detecting distant relationships. In Dayhoff, M. O. (ed.) Atlas of Protein Sequence and Structure, National Biomedical Research Foundation, Washington D.C. Vol. 5, Suppl. 3, pp. 345-358; Hein J., 1990, Unified Approach to Alignment and Phylogenes pp. 626-645 Methods in Enzymology vol. 183, Academic Press, Inc., San Diego, Calif.; Higgins, D. G. and Sharp, P. M., 1989, CABIOS 5:151-153; Myers, E. W. and Muller W., 1988, CABIOS 4:11-17; Robinson, E. D., 1971, Comb. Theor. 11:105; Santou, N., Nes, M., 1987, Mol. Biol. Evol. 4:406-425; Sneath, P. H. A. and Sokal, R. R., 1973, Numerical Taxonomy the Principles and Practice of Numerical Taxonomy, Freeman Press, San Francisco, Calif.; Wilbur, W. J. and Lipman, D. J., 1983, Proc. Natl. Acad. Sci. USA 80:726-730.

Preferably, the “percentage of sequence identity” is determined by comparing two optimally aligned sequences over a window of comparison of at least 20 positions, wherein the portion of the polypeptide sequence in the comparison window may comprise additions or deletions (i.e. gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical amino acid residue occurs in both sequences to yield the number of matched positions, dividing the number of matched positions by the total number of positions in the reference sequence (i.e. the window size) and multiplying the results by 100 to yield the percentage of sequence identity.

Variants also include conjugate comprising any of the insulin protein described herein, e.g., an insulin chemically derivatized with a poly(ethylene glycol) and/or a fatty acid.

Variants also include functional equivalent variants. The biological activities of the variants may be tested using methods known in the art and methods described herein. In some variations, functional equivalent variants have at least about any of 50%, 60%, 70%, 75%, 80%, 85%, 90%, or 95% of activity as compared to a wild-type insulin with respect to one or more of the biological assays described herein (or known in the art). The potencies of insulin can be determined by the mouse convulsion assay, by blood glucose lowering effect and diaphragm glycogen increase in fasted rats in vivo, or by stimulation of “C-glucose metabolism in fat cells and binding to anti-insulin serum in vitro. See F. Märki and W. Albrecht, Diabetologia, 13(4):293-295 (1977).

### Liquid Insulin Formulations

Arginine and proline at effectively high concentrations are used in the refolding process of denatured protein by urea or guanidine. However, proline is found to diminish refolding efficiency although it improves solubility of denatured proteins. Therefore, proline is no longer used as widely as arginine for protein refolding. Without wishing to be bound by any theories, it is postulated that proline at effectively high concentrations has the ability to deter denatured proteins from conformational change in solutions, and also to deter those rightly-folded proteins from un-desired conformational changes which in turn may lead to protein fibrillations, aggregations and precipitations. Such proteins include but not limited to examples like calcitonin, PTH, and monomeric insulin. Arginine and proline have been used in protein formulations at lower concentrations. This invention demonstrates that arginine and proline at higher concentrations can stabilize monomeric insulin in solution. Suitable arginine concentration is 0.4 mol/L or higher, preferably 0.5 mol/L or higher. Suitable proline concentration is 0.9 mol/L or higher, preferably 1.5 mol/L or higher.

In one aspect, the invention provides a stable liquid insulin composition comprising a monomeric insulin and proline, a proline derivative, and/or arginine. In some embodiments, the stable liquid insulin composition comprises proline. In some embodiments, the stable liquid insulin composition comprises a proline derivative. Examples of proline derivatives include, but are not limited to, N-methylproline, 2-methylproline, 3-methylproline, 5-methylproline, N-ethylproline, 2-ethylproline, 3-ethylproline, and 5-ethylproline. In some embodiments, the concentration of proline or a proline derivative in the composition is about 1 to about 7 mol/L. In some embodiments, the concentration of proline or a proline derivative in the composition is about 0.9 to about 7 mol/L, about 0.9 to about 6 mol/L, about 0.9 to about 5 mol/L, about 0.9 to about 4 mol/L, about 0.9 to about 3 mol/L, about 0.9 to about 2.5 mol/L, about 0.9 to about 2 mol/L, about 0.9 to about 1.5 mol/L, about 1 to about 3 mol/L, about 1 to about 2 mol/L, about 1.5 to about 2.5 mol/L, about 2 to about 5 mol/L, about 2 to about 4 mol/L, about 2 to about 3 mol/L, about 3 to about 7 mol/L, about 3 to about 6 mol/L, about 3 to about 5 mol/L, about 3 to about 4 mol/L, about 4 to about 7 mol/L, about 4 to about 6 mol/L, about 4 to about 5 mol/L, about 5 to about 7 mol/L, about 5 to about 6 mol/L, or about 6 to about 7 mol/L. In some embodiments, the stable liquid insulin composition comprises arginine. In some embodiments, the concentration of arginine in the composition is about 0.4 mol/L or higher, or about 0.5 mol/L or higher. In some embodiments, the concentration of arginine in the composition is about 0.4 to about 1.0 mol/L, about 0.4 to about 2.0 mol/L, about 0.5 to about 1.0 mol/L, about 0.5 to about 2.0 mol/L, about 0.5 to about 1.5 mol/L, or about 1.0 to about 2.0 mol/L.

In some embodiments, the stable liquid insulin composition comprises a monomeric insulin and proline and/or arginine. In some embodiments, the monomeric insulin is a human insulin, a porcine insulin, a bovine insulin, an insulin analog, an insulin derivative, or a combination thereof in any ratio. In some embodiments, the insulin is a zinc-free human insulin, a zinc-free porcine insulin, a zinc-free bovine insulin, a zinc-free insulin analog, a zinc-free insulin derivative, or a combination thereof in any ratio. In some embodiments, the monomeric insulin is an insulin analog. Insulin analogs include those peptides with alterations of amino acid sequences such as insulin lispro, insulin aspart, insulin glulisine, or a derivative thereof. In some embodiments, the insulin is an insulin derivative such as an insulin chemically modified with other chemical reagents such as a poly(ethylene glycol) (PEG) and/or a fatty acid.

In some embodiments, the insulin may be extracted from a living tissue. In other embodiments, the monomeric insulin is chemically synthesized or prepared using a recombinant technology. These technologies are known in the art.

In some embodiments, the stable liquid insulin composition comprises about 0.01 mg/mL to about 20 mg/mL of monomeric insulin. In some embodiments, the stable liquid insulin composition comprises about 2 mg/mL to about 4 mg/mL of monomeric insulin. In some embodiments, the stable liquid insulin composition comprises about 0.01 mg/mL to about 0.05 mg/mL, about 0.01 mg/mL to about 0.1 mg/mL, about 0.01 mg/mL to about 0.5 mg/mL, about 0.01 mg/mL to about 1 mg/mL, about 0.01 mg/mL to about 2 mg/mL, about 0.01 mg/mL to about 5 mg/mL, about 0.01 mg/mL to about 10 mg/mL, about 0.1 mg/mL to about 0.5 mg/mL, about 0.1 mg/mL to about 1 mg/mL, about 0.1 mg/mL to about 2 mg/mL, about 0.1 mg/mL to about 5 mg/mL, about 0.1 mg/mL to about 10 mg/mL, about 0.1 mg/mL to about 20 mg/mL, about 1 mg/mL to about 2 mg/mL, about 1 mg/mL to about 5 mg/mL, about 1 mg/mL to about 10 mg/mL, about 1 mg/mL to about 20 mg/mL, about 2 mg/mL to about 5 mg/mL, about 2 mg/mL to about 10 mg/mL, about 2 mg/mL to about 20 mg/mL, about 4 mg/mL to about 10 mg/mL, about 4 mg/mL to about 20 mg/mL, or about 10 mg/mL to about 20 mg/mL of monomeric insulin. In some embodiments, the stable liquid insulin composition comprises about 2 mg/mL, about 4 mg/mL, about 5 mg/mL, about 10 mg/mL, or about 20 mg/mL of monomeric insulin.

The invention also provides a class of small molecules that can further improve chemical stability of monomeric insulin in solutions in the presence of proline. This class of molecules bears a carbonyl group that reversibly forms Schiff bond with amine groups of proteins. This class of small molecules is acetone and acetone analogs, including but not limited to methyl ethyl ketone, propyl methyl ketone, isopropyl methyl ketone, pyruvic acid, glyoxalic acid, alpha-keto butanoic acid, alpha-keto glutaric acid, acetoacetic acid, pyridoxal, pyridoxal phosphate, and other ketone or aldehyde compounds that do not have hydroxyl group at the carbon atom next to carbonyl which may leads to Maillard reaction or similar chemical reactions.

In some embodiments, the stable liquid insulin composition comprising a monomeric insulin and proline and/or arginine further comprises a stabilizing agent which is a compound capable of forming a Schiff bond with an amino group. The stabilizing agent may be selected from the group consisting of acetone, pyruvic acid, glyoxalic acid, alpha-ketobutyric acid, alpha-ketoglutaric acid, acetoacetic acid, pyridoxal, and pyridoxal pyrophosphate. In some embodiments, the stable liquid insulin composition further comprises acetone. In some embodiments, the stable liquid insulin composition contains about 0 to 50 mg/mL of the stabilizing agent. In some embodiments, the stable liquid insulin composition contains about 0 to about 25 mg/mL, about 0 to about 10 mg/mL, about 0 to about 5 mg/mL, about 0 to about 3 mg/mL, about 0 to about 1 mg/mL, about 5 to about 45 mg/mL, about 5 to about 25 mg/mL, about 10 to about 50 mg/mL, about 10 to about 30 mg/mL, about 10 to about 20 mg/mL, or about 20 to about 40 mg/mL of the stabilizing agent, e.g. acetone.

In some embodiments, the stable liquid insulin composition further comprises a pharmaceutically acceptable excipient, such as glycerol (e.g., 0-50 mg/mL) and/or a phenolic compound (e.g., phenol and/or m-cresol). The composition may contain at least 3 moles of the phenolic compound for every 6 moles of the insulin monomer (i.e., the molar concentration of the phenolic compound is at least 50% of the molar concentration of the insulin monomer in the composition), up to a sufficient level for hygienic purposes (e.g., about 1 mg/mL, about 2.5 mg/mL, about 5 mg/mL, or about 10 mg/mL). In some embodiment, the composition contains about 0 to about 40 mg/mL, about 0 to about 20 mg/mL, about 0 to about 10 mg/mL, about 0 to about 5 mg/mL , about 10 to about 50 mg/mL, about 10 to about 40 mg/mL, about 15 to about 35 mg/mL, or about 16 to about 32 mg/mL of glycerol. In some embodiment, the composition contains about 0 to about 10 mg/mL, about 0 to about 5 mg/mL, about 0 to about 4 mg/mL, about 0 to about 3 mg/mL , about 1 to about 5 mg/mL, about 1 to about 4 mg/mL, about 1 to about 3 mg/mL, or about 2 to about 3 mg/mL of the phenolic compound, e.g. in-cresol or phenol. In some embodiment, the composition contains about 0 to about 40 mg/mL, about 0 to about 20 mg/mL, about 0 to about 10 mg/mL, about 0 to about 5 mg/mL , about 10 to about 50 mg/mL, about 10 to about 40 mg/mL, about 15 to about 35 mg/mL, or about 16 to about 32 mg/mL of glycerol, and about 0 to about 10 mg/mL, about 0 to about 5 mg/mL, about 0 to about 4 mg/mL, about 0 to about 3 mg/mL , about 1 to about 5 mg/mL, about 1 to about 4 mg/mL, about 1 to about 3 mg/mL, or about 2 to about 3 mg/mL of the phenolic compound, e.g. in-cresol or phenol.

In some embodiments, the invention provides a stabilized solution of monomeric insulin. This solution contains insulin, stabilizing reagents including proline or a proline derivative (e.g., 1-7 mol/L or 0.9-7 mol/L), pharmaceutically acceptable excipients, and a pH range between 6.0 and 8.0, preferably between 6.8 and 7.8.

The stabilizing reagents in the monomeric insulin solution of the invention also include compounds (0-50 mg/ml) that forms Schiff bond with amines. Preferably, those compounds are selected from but not limited to acetone, pyruvic acid, glyoxalic acid, alpha-ketobutyric acid, glutaric acid, acetoacetic acid, pyridoxal, and pyridoxal pyrophosphate.

In some embodiments, the stable liquid insulin composition further comprises a phosphate buffer. The concentration of the phosphate buffer may be about 5 mM, about 10 mM, about 15 mM, or about 20 mM. In some embodiments, the stable liquid insulin composition further comprises about 1 mM, about 3 mM, about 5 mM, or about 10 mM of EDTA or ETPA. In some embodiments, the stable liquid insulin composition further comprises about 1 mM to about 3 mM, about 1 mM to about 5 mM, about 1 mM to about 10 mM, about 3 mM to about 5 mM, about 1 mM to about 10 mM, or about 5 mM to about 10 mM of EDTA or ETPA.

### Medicaments

In another aspect, the invention provides a medicament comprising a monomeric insulin, proline and/or arginine, and a pharmaceutically acceptable excipient, wherein the medicament is a liquid suitable for administration in a subject in need thereof by nasal and/or pulmonary inhalation, syringe injection, insulin pen and/or cartridge, or insulin pump. In some embodiments, the medicament comprises a monomeric insulin and proline.

In some embodiments, the concentration of proline or a proline derivative in the composition is about 1 to about 7 mol/L. In some embodiments, the concentration of proline or a proline derivative in the composition is about 0.9 to about 7 mol/L, about 0.9 to about 6 mol/L, about 0.9 to about 5 mol/L, about 0.9 to about 4 mol/L, about 0.9 to about 3 mol/L, about 0.9 to about 2.5 mol/L, about 0.9 to about 2 mol/L, about 0.9 to about 1.5 mol/L, about 1 to about 3 mol/L, about 1 to about 2 mol/L, about 1.5 to about 2.5 mol/L, about 2 to about 5 mol/L, about 2 to about 4 mol/L, about 2 to about 3 mol/L, about 3 to about 7 mol/L, about 3 to about 6 mol/L, about 3 to about 5 mol/L, about 3 to about 4 mol/L, about 4 to about 7 mol/L, about 4 to about 6 mol/L, about 4 to about 5 mol/L, about 5 to about 7 mol/L, about 5 to about 6 mol/L, or about 6 to about 7 mol/L. In some embodiments, the medicament comprises a monomeric insulin and arginine. In some embodiments, the concentration of arginine in the composition is about 0.4 mol/L or higher, or about 0.5 mol/L or higher. Examples of proline derivatives include, but are not limited to, N-methylproline, 2-methylproline, 3-methylproline, 5-methylproline, N-ethylproline, 2-ethylproline, 3-ethylproline, and 5-ethylproline.

In some embodiments, the liquid insulin medicament comprises a monomeric insulin and proline or arginine. In some embodiments, the monomeric insulin is a human insulin, a porcine insulin, a bovine insulin, an insulin analog, an insulin derivative, or a combination thereof in any ratio. In some embodiments, the insulin is a zinc-free human insulin, a zinc-free porcine insulin, a zinc-free bovine insulin, a zinc-free insulin analog, a zinc-free insulin derivative, or a combination thereof in any ratio. In some embodiments, the monomeric insulin is generated by means of mixing a zinc-chelating reagent with a zinc human insulin, a zinc porcine insulin, a zinc bovine insulin, a zinc insulin analog, a zinc insulin derivative, or a combination thereof in any ratio. In some embodiments, the monomeric insulin is an insulin analog such as insulin lispro, insulin aspart, insulin glulisine, or a derivative thereof. In some embodiments, the insulin is an insulin derivative such as an insulin derivatized chemically with a poly(ethylene glycol) (PEG) and/or a fatty acid. In some embodiments, the insulin may be extracted from a living tissue. In other embodiments, the monomeric insulin is chemically synthesized or prepared using a recombinant technology. In some embodiments, the medicament comprises about 0.01 mg/mL to about 20 mg/mL of monomeric insulin. In some embodiments, the medicament comprises about 2 mg/mL to about 4 mg/mL of monomeric insulin. In some embodiments, the medicament comprises about 0.01 mg/mL to about 0.05 mg/mL, about 0.01 mg/mL to about 0.1 mg/mL, about 0.01 mg/mL to about 0.5 mg/mL, about 0.01 mg/mL to about 1 mg/mL, about 0.01 mg/mL to about 2 mg/mL, about 0.01 mg/mL to about 5 mg/mL, about 0.01 mg/mL to about 10 mg/mL, about 0.1 mg/mL to about 0.5 mg/mL, about 0.1 mg/mL to about 1 mg/mL, about 0.1 mg/mL to about 2 mg/mL, about 0.1 mg/mL to about 5 mg/mL, about 0.1 mg/mL to about 10 mg/mL, about 0.1 mg/mL to about 20 mg/mL, about 1 mg/mL to about 2 mg/mL, about 1 mg/mL to about 5 mg/mL, about 1 mg/mL to about 10 mg/mL, about 1 mg/mL to about 20 mg/mL, about 2 mg/mL to about 4 mg/mL of monomeric insulin. In some embodiments, the medicament comprises about 2 mg/mL to about 5 mg/mL, about 2 mg/mL to about 10 mg/mL, about 2 mg/mL to about 20 mg/mL, about 4 mg/mL to about 10 mg/mL, about 4 mg/mL to about 20 mg/mL, or about 10 mg/mL to about 20 mg/mL of monomeric insulin. In some embodiments, the medicament comprises about 2 mg/mL, about 4 mg/mL, about 5 mg/mL, about 10 mg/mL, or about 20 mg/mL of monomeric insulin.

In some embodiments, the liquid insulin medicament comprises a pharmaceutically acceptable excipient, such as glycerol (0-50 mg/mL) and/or a phenolic compound (e.g., phenol and/or m-cresol). The medicament may contain at least 3 moles of the phenolic compound for every 6 moles of the insulin monomer, up to a sufficient level for hygienic purposes (e.g., about 1 mg/mL, about 2 mg/mL, about 3 mg/mL, about 5 mg/mL, or about 10 mg/mL). In some embodiment, the medicament contains about 0 to 40 mg/mL, about 0 to about 20 mg/mL, about 0 to about 10 mg/mL, about 0 to about 5 mg/mL , about 10 to about 50 mg/mL, about 10 to about 40 mg/mL, about 15 to about 35 mg/mL, or about 16 to about 32 mg/mL of glycerol. In some embodiment, the medicament contains about 0 to about 10 mg/mL, about 0 to about 5 mg/mL, about 0 to about 4 mg/mL, about 0 to about 3 mg/mL, about 1 to about 5 mg/mL, about 1 to about 4 mg/mL, about 1 to about 3 mg/mL, or about 2 to about 3 mg/mL of the phenolic compound, e.g. m-cresol or phenol. In some embodiment, the medicament contains about 0 to about 40 mg/mL, about 0 to about 20 mg/mL, about 0 to about 10 mg/mL, about 0 to about 5 mg/mL , about 10 to about 50 mg/mL, about 10 to about 40 mg/mL, about 15 to about 35 mg/mL, or about 16 to about 32 mg/mL of glycerol and about 0 to about 10 mg/mL, about 0 to about 5 mg/mL, about 0 to about 4 mg/mL, about 0 to about 3 mg/mL , about 1 to about 5 mg/mL, about 1 to about 4 mg/mL, about 1 to about 3 mg/mL, or about 2 to about 3 mg/mL of the phenolic compound, e.g. m-cresol or phenol.

In some embodiments, the liquid insulin medicament comprising a monomeric insulin and proline and/or arginine further comprises a stabilizing agent which is a compound capable of forming a Schiff bond with an amino group. The stabilizing agent may be selected from the group consisting of acetone, pyruvic acid, glyoxalic acid, alpha-ketobutyric acid, alpha-ketoglutaric acid, acetoacetic acid, pyridoxal, and pyridoxal pyrophosphate. In some embodiments, the stable liquid insulin composition further comprises acetone. In some embodiments, the stable liquid insulin composition contains about 0 to about 50 mg/ml of the stabilizing agent. In some embodiments, the medicament contains about 0 to about 25 mg/mL, about 0 to about 10 mg/mL, about 0 to about 5 mg/mL, about 0 to about 3 mg/mL, about 0 to about 1 mg/mL, about 5 to about 45 mg/mL, about 5 to about 25 mg/mL, about 10 to about 50 mg/mL, about 10 to about 30 mg/mL, about 10 to about 20 mg/mL, or about 20 to about 40 mg/mL of the stabilizing agent, e.g. acetone.

In some embodiments, the liquid insulin medicament further comprises a phosphate buffer. The concentration of the phosphate buffer may be about 5 mM, about 10 mM about 15 mM, or about 20 mM. In some embodiments, the medicament further comprises about 1 mM, about 3 mM, about 5 mM, or about 10 mM of EDTA or ETPA. In some embodiments, the liquid insulin medicament further comprises about 1 mM to about 3 mM, about 1 mM to about 5 mM, about 1 mM to about 10 mM, about 3 mM to about 5 mM, about 1 mM to about 10 mM, or about 5 mM to about 10 mM of EDTA or ETPA.

The invention also provides a stabilized solution of monomeric insulin that may be used as a therapeutic reagent by means of nasal and/or pulmonary inhalation, syringe injection, insulin pen and/or cartridge, insulin pump. This formulation contains insulin, stabilizing reagents including proline or its derivatives (0.9-7 mol/L), pharmaceutically acceptable excipients, and a pH range between 6.0 and 8.0, preferably between 6.8 and 7.8.

In another embodiment, the invention provides a class of stabilizing reagents for a monomeric insulin solution that may be used as a therapeutic reagent by means of nasal and/or pulmonary inhalation, syringe injection, insulin pen and/or cartridge, insulin pump. This class of stabilizing reagents includes compounds (0-50 mg/mL) that forms Schiff bond with amines. Preferably, those compounds are selected from but not limited to acetone, pyruvic acid, glyoxalic acid, alpha-ketobutyric acid, glutaric acid, acetoacetic acid, pyridoxal, and pyridoxal pyrophosphate.

In another aspect, the invention provides a stabilized therapeutic formulation of monomeric insulin that prevents the on-set of hyperglycemic effects faster than zinc-associated hexameric insulin, i.e., the monomeric insulin formulation of the invention lowers blood glucose concentration faster than zinc-associated hexameric insulin. This formulation comprises insulin, stabilizing reagents including proline or its derivatives (0.9-7 mol/L), pharmaceutically acceptable excipients, and has a pH range between 6.0 and 8.0, preferably between 6.8 and 7.8.

The invention further provides a class of stabilizing reagents in the stabilized therapeutic formulation of monomeric insulin that lowers the blood glucose level faster than zinc-associated hexameric insulin. This class of stabilizing reagents of monomeric insulin in this invention includes compounds (0-50 mg/ml) that forms Schiff bond with amines. Preferably, those compounds are selected from but not limited to acetone, pyruvic acid, glyoxalic acid, alpha-ketobutyric acid, glutaric acid, acetoacetic acid, pyridoxal, and pyridoxal pyrophosphate.

Preferably, the designated insulin is selected from human insulin, porcine or bovine insulin, insulin analogs, insulin derivatives and their combination at various ratios. The insulin can be those extracted from living tissues, or chemically synthesized, or prepared using recombinant technology. Insulin analogs include those peptides with alterations of amino acid sequences such as insulin lispro, insulin aspart, and insulin glulisine. Insulin derivatives include those chemically modified with other chemical reagents such as poly(ethylene glycol) and/or fatty acids.

Preferably, the designated insulin is selected from zinc-free human insulin, zinc-free porcine or bovine insulin, zinc-free insulin analogs, zinc-free insulin derivatives and their combination at various ratios. The insulin can be those extracted from living tissues, or chemically synthesized, or prepared using recombinant technology. Insulin analogs include those peptides with alterations of amino acid sequences such as insulin lispro, insulin aspart, and insulin glulisine. Insulin derivatives include those chemically modified with other chemical reagents such as poly(ethylene glycol) and/or fatty acids.

Also provided is a kit, comprising a medicament described herein and a container (e.g. a nasal and/or pulmonary inhaler, an injection syringe, an insulin pen and/or cartridge, or an insulin pump) for holding the medicament. Thus the container may be a nasal and/or pulmonary inhaler, an injection syringe, an insulin pen and/or cartridge, or an insulin pump. The kit may further comprise instructions for administrating the medicament in a subject in need thereof by means of nasal and/or pulmonary inhalation, syringe injection, insulin pen and/or cartridge, or insulin pump.

This invention reveals that proline and arginine at high concentrations can reduce aggregation/precipitation of peptides or proteins such as monomeric insulin that readily fibrillizes to form precipitates. This invention also reveals proline at high concentration deters insulin from formation of covalent dimers and/or higher oligomers. This invention further reveals that acetone also helps reducing formation of covalent dimers and/or higher oligomers of insulin. This invention finally reveals that combination of proline, acetone and other ingredients leads to a stabilized monomeric insulin solution that lowers the blood glucose level faster than zinc-insulin hexamer.

The therapeutic reagent of monomeric insulin solution has the advantages of reduced covalent high molecular weight degradation side-product and diminished insulin precipitates of fibrillation.

### Methods

In another aspect, the invention provides a method of making a stable monomeric insulin composition comprising: (a) mixing a zinc insulin in a solution with a zinc chelating reagent, wherein the molar ratio of the zinc chelating reagent to zinc is from about 1:1 to about 100:1; and (b) adding proline and/or arginine to the solution formed in step (a) to form a stabilized insulin solution. In some embodiments, the pH range of the stabilized insulin solution is about 6.0 to 8.0, preferably about 6.8 to 7.8. In some embodiments, the zinc insulin is a zinc human insulin, a zinc porcine or bovine insulin, a zinc insulin analog, a zinc insulin derivative, or a combination thereof in any ratio. In some embodiments, the zinc chelating reagent is EDTA or ETPA. In some embodiments, the zinc insulin is extracted from a living tissue, chemically synthesized, or prepared using a recombinant technology. In some embodiments, the stabilized insulin solution contains about 0.9 to about 7 mol/L of proline and/or about 0.4 to about 2.0 mol/L of arginine. In some embodiments, the method further comprises adding acetone to the solution formed in step (a) or the stabilized insulin solution formed in step (b).

In some embodiments, the designated monomeric insulin is generated in-situ by means of mixing zinc-insulin and chelating reagents in solution with pH range between 6.0 and 8.0.

In some embodiments, the designated zinc-insulin is selected from zinc human insulin, zinc porcine or bovine insulin, zinc insulin analogs, zinc insulin derivatives and their combination at various ratios. The zinc-insulin can be those extracted from living tissues, or chemically synthesized, or prepared using recombinant technology. Insulin analogs include those peptides with alterations of amino acid sequences such as insulin lispro, insulin aspart, and insulin glulisine. Insulin derivatives include those chemically modified with other chemical reagents such as poly(ethylene glycol) and/or fatty acids.

Preferably, the zinc-chelating reagent is selected from but not limited to EDTA (ethylenediamine tetraacetic acid), ETPA (diethylenetriamine pentaacetic acid) and their analogs, at ratio of about 1 to about 100 chelating molecules for each zinc ion.

In some embodiments, the method may further comprise a step of adding one or more pharmaceutically acceptable excipients to the solution formed in step (a) and/or (b). Preferably, the excipient also includes glycerol (0-50 mg/ml), and at least 3 molecules of phenolic compounds for every 6 insulin molecules. Preferably, the phenolic compound is selected from phenol and m-cresol.

In another aspect, the invention provides a method for treating hyperglycemia in a subject in need thereof comprising administering to the subject an effective amount of a stable liquid insulin composition described herein or a liquid insulin medicament described herein.

In some embodiments, the stable liquid insulin composition or the liquid insulin medicament is administered by nasal and/or pulmonary inhalation, syringe injection, an insulin pen and/or cartridge, an insulin pump, or other suitable means of administration as determined by a person skilled in the art.

The stabilized solution of monomeric insulin can be used as a therapeutic reagent by means of nasal and/or pulmonary inhalation, syringe injection, insulin pen and/or cartridge, insulin pump or other administration pathway.

This invention reveals that the solution of monomeric insulin containing proline lowers blood glucose concentration in animals (e.g., pigs) faster than zinc-insulin hexamers. Commercial insulin analogs such as insulin glulisine, insulin lispro and insulin aspart are formulated in the form of zinc-insulin hexamers that are more stable than zinc-free form and dissociate into biologically active single-molecule insulin faster than human zinc-insulin and porcine zinc-insulin, and therefore they are used as faster-acting therapeutic drugs. The monomeric insulin solution in this invention does not have the dissociation process from inactive hexamer to active monomer, therefore this invented solution also lowers blood glucose concentration in humans faster than zinc-insulin hexamers.

Some proteins, such as insulin monomer, calcitonin, PTH, human growth hormone, glucagon, all fibrillate to some extent and then form precipitates. Monomeric insulin has good solubility between pH 7 and 8, however it fibrillates to form precipitate over time. The formulation method in this present invention can be applied to this group of peptides and proteins.

In another aspect, the invention provides a method for stabilizing a small protein (e.g. molecular weight under 20,000 Daltons) in solution comprising mixing the protein in solution with one or more small molecule excipients. The method is applicable to small proteins that readily undergo conformational changes, for examples, an insulin monomer, calcitonin, PTH, a human growth hormone, and glucagon. Examples of the small molecule excipients include, but are not limited to proline, proline derivatives (e.g. N-methylproline, 2-methylproline, 3-methylproline, 5-methylproline, N-ethylproline, 2-ethylproline, 3-ethylproline, and 5-ethylproline), arginine, and compounds capable of forming a Schiff bond with an amino group (e.g., acetone, pyruvic acid, glyoxalic acid, alpha-ketobutyric acid, alpha-ketoglutaric acid, acetoacetic acid, pyridoxal, and pyridoxal pyrophosphate). The small molecule excipients stabilize the protein by minimizing protein precipitation caused by denaturing and/or fibrillation.

In some embodiments, the method for stabilizing a small protein (e.g. molecular weight under 20,000 Daltons) in solution comprising mixing the protein in solution with proline or a proline derivative, at a concentration of about 0.9 to about 7 mol/L, about 0.9 to about 6 mol/L, about 0.9 to about 5 mol/L, about 0.9 to about 4 mol/L, about 0.9 to about 3 mol/L, about 0.9 to about 2.5 mol/L, about 0.9 to about 2 mol/L, about 0.9 to about 1.5 mol/L, about 1 to about 3 mol/L, about 1 to about 2 mol/L, about 1.5 to about 2.5 mol/L, about 2 to about 5 mol/L, about 2 to about 4 mol/L, about 2 to about 3 mol/L, about 3 to about 7 mol/L, about 3 to about 6 mol/L, about 3 to about 5 mol/L, about 3 to about 4 mol/L, about 4 to about 7 mol/L, about 4 to about 6 mol/L, about 4 to about 5 mol/L, about 5 to about 7 mol/L, about 5 to about 6 mol/L, or about 6 to about 7 mol/L of proline or a proline derivative.

In some embodiments, the invention provides a method for improving the physical and chemical stability of monomeric insulin in solution, in which proline or a proline derivative is added as the stabilizer at concentrations ranging from 1 to 7 mol/L. In some embodiments, the monomeric insulin in solution is further stabilized by adding 0-50 mg/mL of a chemical compound that can form reversible Schiff bond with amines. The compound may be selected from but not limited to acetone, pyruvate, glyoxalate, alpha-keto butyric acid, alpha-ketoglutaric acid, acetoacetate (oxalacetate), pyridoxal, and pyridoxal phosphate. Preferably, the insulin useful in the method includes human insulin, porcine or bovine insulin, insulin analogs, insulin derivatives and their combination at various ratios. The insulin can be those extracted from living tissues, or chemically synthesized, or prepared using recombinant technology. Insulin analogs include those peptides with alterations of amino acid sequences such as insulin lispro, insulin aspart, insulin glulisine, and other insulin variants. Insulin derivatives include those chemically modified with other chemical reagents such as poly(ethlylene glycol) and/or fatty acids.

The invention provides methods for preparing stable insulin solutions that lower the blood glucose level faster than zinc-associated hexameric insulin. This method may be applied to improve physical and/or chemical stability of other proteins in solutions, especially proteins like monomeric insulin that are known for its quick fibrillation and precipitation in solutions. The chemical stability includes issues of protein degradations such as hydrolysis and formation of covalent dimmers or higher oligomers, the physical stability includes issues of protein aggregate formation and precipitation caused by denaturing and/or fibrillation.

The invention further provides a composition comprising a protein and proline or a proline derivative, wherein the composition is in a liquid form, wherein the protein has a molecular weight of less that 20,000 Daltons, and wherein the proline or proline derivative in the composition has a concentration of about 0.9 mol/L to about 7.0 mol/L and stabilizes the protein by minimizing protein precipitation caused by denaturing and/or fibrillation. In some embodiments, the protein is an insulin monomer, calcitonin, PTH, a human growth hormone, or glucagon. In some embodiments, the composition further comprises a compounds capable of forming a Schiff bond with an amino group (e.g., acetone, pyruvic acid, glyoxalic acid, alpha-ketobutyric acid, alpha-ketoglutaric acid, acetoacetic acid, pyridoxal, and pyridoxal pyrophosphate).

### EXAMPLES

The following Examples are provided to illustrate but not limit the invention.

### Example 1

### Stabilizing Effects of Arginine on Monomeric Insulin in Solutions

Insulin was dissolved in 0.10 N hydrochloric acid (5 μL per milligram of insulin) and made into a series of solutions at its concentration of 4.0 mg/ml with various additives, and the pH was adjusted to pH 7.4 using 0.05 N sodium hydroxide. Samples were kept at room temperature (25° C.) in the dark.

TABLE 1

| | Recombinant human Insulin | | | Recombinant human | |
|---|---|---|---|---|---|
| | Sodium Salt, Zinc Free | | | Insulin containing Zinc | |
| | Sample Number | | | | |
| | 1 | 2 | 3 | 4 | 5 |
| [insulin] | 4 mg/ml | 4 mg/ml | 4 mg/ml | 4 mg/ml | 4 mg/ml |
| [Arginine] | 0 | 0.5M | 0.5M | 0.5M | 0.5M |
| [EDTA] | 0 | 0 | 0 | 0.1 mM | 0.1 mM |
| [Glycerol] | 0 | 0 | 16 mg/ml | 0 | 16 mg/ml |
| [m-Cresol] | 0 | 0 | 2.5 mg/ml | 0 | 2.5 mg/ml |
| 4 days | Precipitate at | No | No | No | No |
| | Bottom | Precipitate | Precipitate | Precipitate | Precipitate |
| 7 days | Precipitate at | No | No | No | No |
| | Bottom | Precipitate | Precipitate | Precipitate | Precipitate |

This example demonstrates that arginine at 0.5 M has ability to deter monomeric insulin from precipitation.

### Example 2

### Stabilizing Effects of Proline on Sodium Insulin in Solutions

Proline (17.36 g) was dissolved in sodium phosphate buffer (50 mM, pH 7.4) to a volume of 50 ml, giving final proline concentration of 3.0 M at pH 7.3.

A stock solution was made in water containing glycerol (320 mg/ml) and m-Cresol (20 mg/ml).

Zinc-free recombinant human insulin (16 mg) was dissolved in 0.10 N hydrochloric acid (70 microliters), and to this insulin was added 6.0 ml of the above proline solution. This stock solution was further made into a series of solutions at its concentration of 2.0 mg/ml with various additives at pH 7.4. Samples were kept at room temperature (25° C.) in the dark.

TABLE 2

| | Recombinant human Insulin Sodium Salt | | |
|---|---|---|---|
| | Sample Number | | Novolog |
| | 1 | 2 | 3 |
| [insulin] | 2 mg/ml | 2 mg/ml | 100 IU/ml |
| [Proline] | 2.4M | 2.4M | 0 |
| [Glycerol] | 0 | 32 mg/ml | 16 mg/ml |
| [m-Cresol] | 0 | 2.0 mg/ml | 2.5 mg/ml |

After six months, there were no precipitates visible at the bottom, and the solutions were all visually clear. As shown in Example 1 above, insulin without proline forms precipitates in a few days.

This example demonstrates that proline at this concentration shows its ability to deter monomeric insulin from precipitation.

### Example 3

### Proline Effect at Various Concentrations

Repeat preparation as in Example 2 at proline concentration of 1.0 M, 1.5 M, 2.4 M and 7.0 M, without glycerol and m-cresol. All samples were stored at room temperature in the dark over six month to find no visible insulin precipitate.

This example confirms the proline effects on insulin's physical stability in solutions.

### Example 4

### Eight Insulin Test Formulations and SEC HPLC Analysis

A group of stock solutions were prepared: proline (6.25 M) in water, EDTA (0.10 M, pH 7.4), glycerol(320 mg/ml) containing m-Cresol (25 mg/ml) in water, sodium insulin(10 mg/ml) in phosphate buffer (25 mM, pH 7.4). Eight formulations were prepared using those stock solutions as in the table below.

TABLE 3

| | Sodium Insulin | | | | Zinc Insulin | | | |
|---|---|---|---|---|---|---|---|---|
| | (Final pH 7.1) | | | | (Final pH 7.4) | | | |
| | Sample Number | | | | | | | |
| | 1 | 2 | 3 | 4 | 5 | 6 | 7 | 8 |
| [Insulin], | 4.0 | 4.0 | 4.0 | 4.0 | 4.0 | 4.0 | 4.0 | 4.0 |
| mg/ml | | | | | | | | |
| [Proline], M | 0 | 0 | 2.5 | 2.5 | 0 | 0 | 2.5 | 2.5 |
| [Glycerol], | 32 | 32 | 32 | 32 | 32 | 32 | 32 | 32 |
| mg/ml | | | | | | | | |
| [m-Cresol], | 2.5 | 2.5 | 2.5 | 2.5 | 2.5 | 2.5 | 2.5 | 2.5 |
| mg/ml | | | | | | | | |
| [EDTA], mM | 0 | 0 | 0 | 0 | 10 | 10 | 10 | 10 |
| [Acetone], | 0 | 3.0 | 0 | 3.0 | 0 | 3.0 | 0 | 3.0 |
| mg/ml | | | | | | | | |
| [Phosphate], | 10 | 10 | 10 | 10 | 10 | 10 | 10 | 10 |
| mM | | | | | | | | |

All the eight formulations were kept at room temperature in the dark. At times, aliquots were withdrawn for HPLC analysis (Injection volume: 0.10 ml).

During the storage, covalent insulin dimers were quantified by SEC HPLC, and the results are summarized below.

Column: Protein Pak, 125 Å, Waters, WAT084601

Mobile Phase: 20% Acetonitrile, 15% acetic acid, 65% water

Flow rate: 0.5 ml/ml

Detection: 260 nm

TABLE 4

| | Sample Number | | | | | | | |
|---|---|---|---|---|---|---|---|---|
| | 1 | 2 | 3 | 4 | 5 | 6 | 7 | 8 |
| 12 | 1.4% | 1.1% | 0.78% | ~0% | 1.4% | 1.4% | 0.53% | ~0% |
| weeks | | | | | | | | |
| 20 | 2.5% | 2.1% | 1.2% | ~0% | 2.7% | 3.3% | 0.72% | ~0% |
| weeks | | | | | | | | |
| 35 | 6.2% | 5.4% | 2.2% | 0.75% | 7.1% | 7.6% | 1.5% | ~0% |
| weeks | | | | | | | | |

This example shows that proline alone or proline together with acetone reduces formation of high molecular weight insulin degradation side products, in both cases of zinc-free insulin and zinc-insulin with EDTA.

### Example 5

### Proline and Acetone Effects on Chemical Stability of Porcine Insulin

Six test formulations were prepared at proline concentrations of 0, 1.6 M and 2.8 M with acetone concentrations at 0 and 3 mg/ml, as listed in the following table. Those test formulations were stored at room temperature in the dark. At times, aliquots were withdrawn to be analyzed on C18 reverse-phase HPLC to measure total degradation products, as summarized in the table below.

TABLE 5

| | | Sample Number | | | | | |
|---|---|---|---|---|---|---|---|
| | | 1 | 2 | 3 | 4 | 5 | 6 |
| Composition | [Insulin], mg/ml | 4.0 | 4.0 | 4.0 | 4.0 | 4.0 | 4.0 |
| | [Proline], M | 0 | 0 | 0.93 | 0.93 | 1.56 | 1.56 |
| | [Glycerol], | 32 | 32 | 32 | 32 | 32 | 32 |
| | mg/ml | | | | | | |
| | [m-Cresol], | 2.5 | 2.5 | 2.5 | 2.5 | 2.5 | 2.5 |
| | mg/ml | | | | | | |
| | [EDTA], mM | 10 | 10 | 10 | 10 | 10 | 10 |
| | [Acetone], | 0 | 3.0 | 0 | 3.0 | 0 | 3.0 |
| | mg/ml | | | | | | |
| | [Phosphate], | 10 | 10 | 10 | 10 | 10 | 10 |
| | mM | | | | | | |
| Stability | Week 0 | 6.4% | 8.0% | 6.4 | 8.0 | 6.7 | 8.2 |
| | Week 4 | 11.6% | 13.8% | 9.8% | 9.8% | 10.7% | 10.0% |
| | Week 8 | 14.3% | 16.7% | 11.6% | 11.7% | 12.0% | 12.2% |
| | Total Change | +7.9% | +8.7% | +5.2% | +3.7% | +5.3% | +4.0% |

As shown in the table, proline reduces total amount of degradation side products over a period of 8 weeks. Addition of acetone further enhances the stabilizing effect of proline.

### Example 6

### Blood Glucose Lowering Activity in Pigs

Two formulations were prepared from recombinant human zinc insulin and zinc-free insulin in solutions at pH 7.2.

TABLE 6

| Samples | Formulation A | Formulation B |
|---|---|---|
| Insulin Type | Zinc-free Insulin | Zinc Insulin |
| [insulin] | 2 mg/ml | 2 mg/ml |
| [EDTA] | 0 | 20 mM |
| [Proline] | 2.4M | 2.4M |
| [Glycerol] | 16 mg/ml | 16 mg/ml |
| [m-Cresol] | 3.2 mg/ml | 3.2 mg/ml |
| [Sodium Phosphate] | 10 mM | 20 mM |

Formulation A and B were tested for their activities in lowering the blood glucose, using commercial Novolin and Novolog as references. The tests were performed in four pigs (25 kg) at dose of one unit per kilograms. Insulins were injected subcutaneously on their necks, and at intervals, blood samples were withdrawn from ear for glucose concentration measurement (Monroe method).

TABLE 7

| | Blood Glucose (mmol/L) | | | | | | | | |
|---|---|---|---|---|---|---|---|---|---|
| Time After | | | Average | | | | | | |
| Injection | | | before | | | | | | |
| (minutes) | −30 | −15 | Injection | 20 | 30 | 40 | 60 | 120 | 180 |
| Novolin R | 1.6 | 1.8 | 1.7 | 1.2 | 1.1 | 1.2 | 0.7 | 0* | 0.7 |
| Novolog | 1.9 | 1.9 | 1.9 | 0* | 0* | 0* | 0* | — | — |
| Formulation | 1.1 | 1.3 | 1.2 | 0.6 | 0* | 0* | 0* | — | — |
| A | | | | | | | | | |
| Formulation | 1.4 | 1.7 | 1.55 | 0.7 | 0* | 0* | — | — | — |
| B | | | | | | | | | |

*The blood glucose level is below the detection limit.

This example demonstrates that both formulation A and B lower blood glucose to detection limit faster than Novolin R that is a formulation of zinc-insulin hexamer.

Although the foregoing invention has been described in some detail by way of illustration and example for purposes of clarity of understanding, the descriptions and examples should not be construed as limiting the scope of the invention.

All references, publications, and patent applications disclosed herein are hereby incorporated by reference in their entirety.

1. A composition comprising a monomeric insulin and proline or a proline derivative, wherein the composition is in a liquid form.

2. The composition of claim 1, wherein the insulin is a human insulin, a porcine insulin, a bovine insulin, an insulin analog, an insulin derivative, or a combination thereof.

3. The composition of claim 1, wherein the insulin is a zinc-free human insulin, a zinc-free porcine insulin, a zinc-free bovine insulin, a zinc-free insulin analog, a zinc-free insulin derivative, or a combination thereof.

4-10. (canceled)

11. The composition of claim 1, further comprising a stabilizing agent which is a compound capable of forming a Schiff bond with an amino group.

12. The composition of claim 11, wherein the stabilizing agent is selected from the group consisting of acetone, pyruvic acid, glyoxalic acid, alpha-ketobutyric acid, alpha-ketoglutaric acid, acetoacetic acid, pyridoxal, and pyridoxal pyrophosphate.

13-14. (canceled)

15. The composition of claim 1, further comprising a pharmaceutically acceptable excipient.

16. The composition of claim 15, wherein the pharmaceutically acceptable excipient is glycerol and/or a phenolic compound.

17-20. (canceled)

21. The composition of claim , wherein the pH range of the liquid is between about 6.0 and about 8.0.

22-24. (canceled)

25. A composition comprising a monomeric insulin and arginine, wherein the composition is in a liquid form.

26. (canceled)

27. A method of making a stable monomeric insulin composition comprising:
(a) mixing a zinc insulin in a solution with a zinc chelating reagent, wherein the molar ratio of the zinc chelating reagent to zinc is from about 1:1 to about 100:1; and (b) adding proline, a proline derivative, and/or arginine to the solution formed in step (a) to form a stabilized insulin solution.

28-29. (canceled)

30. The method of claim 27, wherein the zinc insulin is selected from the group consisting of a zinc human insulin, a zinc porcine insulin, a zinc bovine insulin, a zinc insulin analog, a zinc insulin derivative, and a combination thereof.

3. (canceled)

34. The method of claim 27, further comprising adding a stabilizing agent to the solution formed in step (a) or the stabilized insulin solution.

35-37. (canceled)

38. The method of claim 27, further comprising adding a pharmaceutically acceptable excipient to solution formed in step (a) or the stabilized insulin solution.

39-43. (canceled)

44. A stable monomeric insulin composition made by the method of claim 27.

45-75. (canceled)

76. A method for stabilizing a protein in solution comprising mixing the protein in solution with one or more small molecule excipients, wherein the one or more small molecule excipient comprise praline or a praline derivative at a concentration of about 0.9 mon to about 7.0 moll,.

77-88. (canceled)
